(19) United States
(12) Patent Application Publication
(10) Pub. No.: US 2012/0253449 A1
(43) Pub. Date: Oct. 4, 2012

(54) Stent

(75) Inventors: Fuh-Yu Chang, Zhubei City (TW); Chia-Chang Liu, New Taipei City (TW); Min-Hua Chen, Taipei City (TW); Po-Chin Liang, Taipei City (TW); Yu-Ting Chang, Taipei City (TW); Kai-Wen Huang, Taipei City (TW); Yi-Chao Hsu, Miaoli County (TW); Hsiao-Chin Cheng, Tainan City (TW); Yu-Ting Fang, New Taipei City (TW); Mei-Shey Lin, Taipei City (TW)

(73) Assignee: NATIONAL TAIWAN UNIVERSITY, Taipei (TW)

(21) Appl. No.: 13/337,688

(22) Filed: Dec. 27, 2011

(30) Foreign Application Priority Data
Apr. 1, 2011 (TW) 100111477

(51) Int. Cl. A61F 2/82

(52) U.S. Cl. 623/1.15

(57) ABSTRACT

The present invention discloses a stent for supporting a sidewall of a lumen which contains a first fluid. The stent comprises a stent body, a film and a photosensitizing layer. The stent body is disposed inside the lumen. The film is adapted to be penetrated by a light and covers the stent body. The photosensitizing layer is disposed on an outer surface of the film for receiving the light to generate a first object, and the film separates the photosensitizing layer and the first fluid. The film and the photosensitizing layer of the present invention can greatly reduce the needed dosage of the photosensitizer while maintaining the effective concentration of the photosensitizer around tumor cells.

### BACKGROUND OF THE INVENTION

1. Field of the Invention

The present invention relates to a stent. More particularly, the present invention relates to a stent which comprises a photosensitizing layer used for photodynamic therapies.

2. Description of the Prior Art

The basic principle of the photo-dynamic therapy (PDT) is to stimulate a photosensitizer by a strong light or a laser, so that a photochemical action will be produced. Then, the cells become toxic for killing the tumor cells or the cancer cells. Wherein, the strong light or the laser shall let the photosensitizer start and penetrate through the human body toward the locations with certain depth.

In prior art, the photosensitizer shall be injected into the human body, so the selection of the photosensitizer is important. For example, the photosensitizer shall be chosen without poison for the human body. Additionally, the photosensitizer shall be selectable to stay in the tumor organization and removed in the short time. However, the dosage of the photosensitizer is high for maintaining the concentration of the photosensitizer so that the photodynamic therapy is expansive.

Ideally, after injecting the photosensitizer into the human body, the staying time of the photosensitizer in the tumor organization is longer than the staying time in the normal organization. Additionally, the photosensitizer can combine with the surface of the tumor organization tightly. After a period of time, the photosensitizer around the tumor will be removed by the lymphatic system to maintain the certain concentration. Then, the tumor organization will be destroyed by the strong light or the laser. In prior art, the high dosage of the photosensitizer is injected into the human body, besides the tumor organization, the normal organization is also with high concentration. Thus, if the skin is irradiated by the light, the photochemical action will be produced at the photosensitizer in the skin cells and the skin cells will be destroyed. For protecting the normal cells from damaging, the patient shall stay in a darkroom and the daily life of the patient will be affected in prior art.

To sum up, it is an important issue about how to limit the photosensitizer around the tumor organization when the light is irradiating.

### SUMMARY OF THE INVENTION

Accordingly, a scope of the invention is to provide a stent. An outer surface of the stent comprises a photosensitizing layer. The stent can support a sidewall of a lumen. At the same time, the stent can diffuse a photosensitizer to the tumor organization. Then, the tumor will be destroyed by irradiating a light.

A scope of the invention is to provide a stent for supporting a sidewall of a lumen. The lumen is used to contain a first fluid. The stent comprises a stent body, a film and a photosensitizing layer. Wherein, the stent body is disposed inside the lumen. The film is adapted to be penetrated by a light and covers the stent body. The photosensitizing layer is disposed on an outer surface of the film, for receiving the light to generate a first object. The film separates the photosensitizing layer and the first fluid.

According to an embodiment, the stent body is a plastic pipe or a metal net pipe. Additionally, the stent further comprises a lubricating layer disposed on the outer surface of the photosensitizing layer. Wherein, the lubricating layer is hydrophilic and biodegradable, the film is hydrophobic. Furthermore, the wavelength of the light received by the photosensitizing layer ranges from 500 nm to 800 nm. The photosensitizing layer comprises a photosensitizer used for photodynamic therapies. The first object generated by the photosensitizing layer is singlet oxygen.

According to another embodiment, the stent further comprises a light source. The light source is fixed with the stent body for providing the light. Additionally, the stent can further comprise an electrical energy receiver. The electrical energy receiver can transmit the electrical energy provided from the outside of the lumen by a wireless power supply technology.

The stent is covered by the film. The photosensitizing layer of the film can greatly reduce the needed dosage of the photosensitizer while maintaining the effective concentration of the photosensitizer around tumor cells. Because the tumor will press the blood vessel or other lumen, the stent shall be used to maintain the circulation of the portion comprising the tumor. Accordingly, the invention is to provide a stent used for photodynamic therapy. The said stent can provide a photosensitizer to the portion of the tumor and protect the lumen from blocking.

The advantage and spirit of the invention may be understood by the following recitations together with the appended drawings.

### BRIEF DESCRIPTION OF THE APPENDED DRAWINGS

FIG. 1A illustrates a schematic diagram of a stent according to an embodiment of the invention.

FIG. 1B illustrates a cross-sectional diagram of part of stent according to an embodiment of the invention.

FIG. 2 illustrates a cross-sectional diagram of a stent according to another embodiment of the invention.

FIG. 3 illustrates a cross-sectional diagram of part of stent according to other embodiment of the invention.

### DETAILED DESCRIPTION OF THE INVENTION

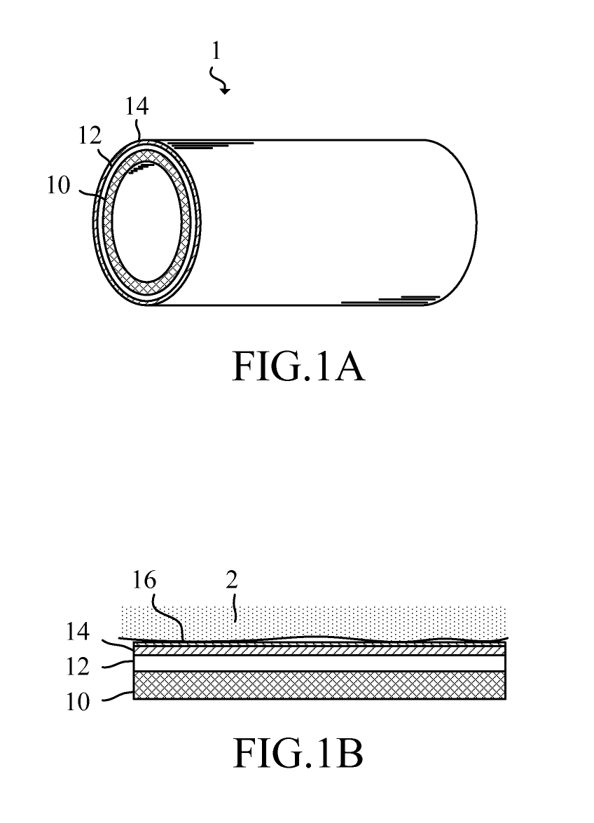

Please refer to FIG. 1A and FIG. 1B. FIG. 1A illustrates a schematic diagram of a stent according to an embodiment of the invention. FIG. 1B illustrates a cross-sectional diagram of part of stent according to an embodiment of the invention. As shown in figures, the stent 1 comprises a stent body 10 a film 12 and a photosensitizing layer 14. The film 12 covers the stent body 10. The photosensitizing layer 14 is disposed on an outer surface of the film 12. Each element of the stent 1 is explained as follows.

The stent body 10 is disposed inside a lumen for supporting a sidewall 12 of a lumen. In practice, the stent body 10 can be a plastic pipe or a metal net pipe. The said plastic pipe or the metal net pipe can be, but not limited to an intravascular stent or other proper stent.

The stent body 10 is covered by the film 12. The film 12 is adapted to be penetrated by a light. The light can be an infrared ray. Generally, the photodynamic therapy is to stimulate a photosensitizer by a light in order to generate a product which is toxic for cells. Thus, the film 12 belongs to the scope of the invention if the film 12 is adapted to be penetrated by a light. For example, the film 12 can be a transparent film or a film adapted to be penetrated by a red light.

In practice, the film 12 can comprise many light shielding patterns to shade the film 12 from the light. The light shielding patterns separate the film 12 into a light transmissive region and a light tight region. The light transmissive region can be penetrated by the light. For example, the film 12 can be added with different colors, the light transmissive region can be transparent and the light tight region can be green for blocking the red light. Therefore, the invention can choose the curing region by irradiating the light and protect the cells from damaging according to the light shielding patterns of the film 12.

The photosensitizing layer 14 receives the light to generate a first object. The film 12 separates the photosensitizing layer 14 and the first fluid of the lumen. Wherein, the first fluid can be blood, bile or other body fluids. In practice, the photosensitizing layer can be stimulated by a strong light or a laser with wavelengths ranging from 500 nm to 800 nm. The wavelengths can be, but not limited to stimulate the photosensitizing layer 14. The red light with a wavelength about 630 nm can be chosen to stimulate the photosensitizing layer 14. After stimulating the photosensitizing layer 14, the first object will be generated in the process from excited state to steady state. The first object can be singlet oxygen and toxic for cells.

To be noticed, the photosensitizing layer 14 can be formed on the film 12 by coating, film coating or other ways. Compared to prior art, the present invention can greatly reduce the needed dosage of the photosensitizer. Additionally, the photosensitizing layer 14 contacts with the sidewall 2 of the lumen tightly, so that the photosensitizer can diffuse into the cells easily. The film 12 obstructs the photosensitizer and the first fluid of the lumen. Thus, the photosensitizer will diffuse toward the tumor cells instead of the normal cells. Accordingly, the present invention can greatly reduce the needed dosage of the photosensitizer and maintain the effective concentration of the photosensitizer around tumor cells.

In addition, the photosensitizing layer 14 can be, but not limited to biodegradable materials or non-biodegradable materials. If the locations of the tumor cells are near the surface of the human body, the photosensitizing layer 14 can be formed by the non-biodegradable materials. Thus, the photosensitizing layer 14 can be irradiated by the light or releases the first object repeatedly. That is to say, the photosensitizing layer 14 comprises the photosensitizer which can be used repeatedly. Oppositely, if the locations of the tumor cells are away the surface of the human body, the photosensitizing layer 14 can be formed by the biodegradable materials. Thus, the photosensitizer can diffuse into the tumor cells deeply. Of cause, the stent 1 can be changed regularly in order to maintain the effect of the photodynamic therapy.

Furthermore, the stent 1 can further comprise a lubricating layer 16 disposed on the outer surface of the photosensitizing layer 14. The lubricating layer 16 is hydrophilic and biodegradable. In practice, if the stent 1 is disposed into the lumen without the lubricating layer 16, the stent 1 cannot be moved toward the predetermined location. When the stent 1 is at the predetermined location, the lubricating layer 16 disappears slowly so that the photosensitizing layer 14 will be revealed and not obstruct the process of the photodynamic therapy. In the invention, the hydrophobic materials can be chosen for the surfaces of the stent body 10, film 12 or photosensitizing layer 14. Thus, the stent 1 is disposed in the lumen steadily. The photosensitizing layer 14 and sidewall 2 of the lumen can be attached tightly.

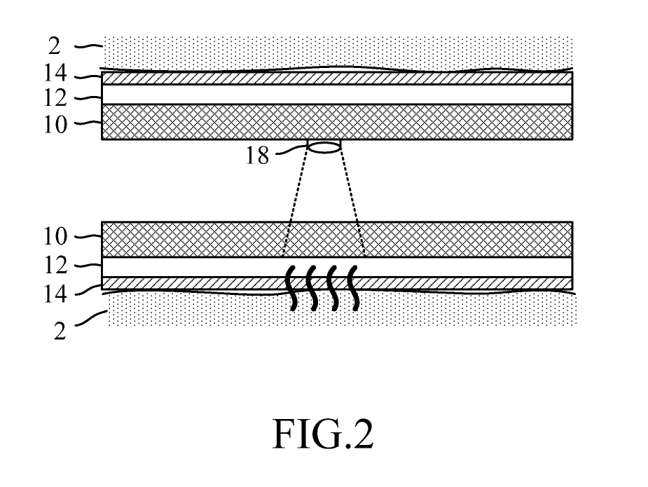

In particular, the invention is to provide an illumination way to match with the stent 1 for photodynamic therapy. Please refer to FIG. 2. FIG. 2 illustrates a cross-sectional diagram of a stent according to another embodiment of the invention. As shown in FIG. 2, the stent 1 further comprises a light source 18 fixed with the stent body 10, for providing the light. In practice, the light source 18 can be, but not limited to a light emitting diode or other light emitting devices. The light source 18 belongs to the scope of the invention if the light source 18 can be used for photodynamic therapy.

Certainly, the embodiment can match the light shielding pattern, so that the light transmissive region can be corresponded to the tumor cells for protecting the normal cells form damaging. Wherein, the light transmissive region can be aligned to the tumor cells by endoscopy, computed tomography or other ways.

Generally, setting power supply equipments on the stent body 10 is difficult, so that the stent 1 can further comprise an electrical energy receiver (not shown in figures). The electrical energy receiver is coupled to the light source 18, for transmitting the electrical energy provided from the outside of the lumen by a wireless power supply technology. Accordingly, the invention does not need to set batteries into human bodies. The power can be transmitted to the light source 18 by a switch outside the human bodies, for controlling whether the light source 18 emits the light or not.

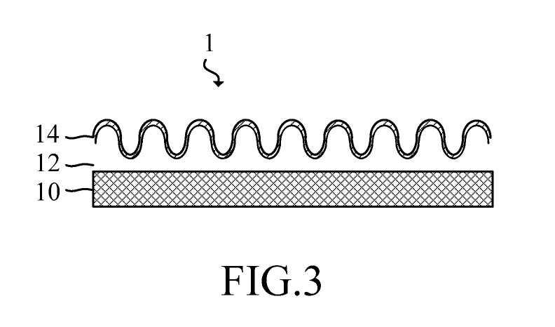

The film 12 can comprises many surface structures. Please refer to FIG. 3. FIG. 3 illustrates a cross-sectional diagram of part of stent according to other embodiment of the invention. As shown in FIG. 3, the film 12 can comprise a porous structure or a ciliate structure. By enlarging the surface area of the film 12, the dosage coated on the photosensitizing layer 14 of the film 12 can be increased. If the photosensitizing layer 14 is a biodegradable material, the stent 1 can release photosensitizer long time at the portion of tumor cells and the service cycle of the stent 1. Additionally, the present invention can adjust the releasing speed of the photosensitizer by the porous structure or the ciliate structure.

To sum up, the tumor will press the blood vessel or other lumen and be fatal to patients, so that the stent shall be used to maintain the circulation of the portion comprising the tumor. Accordingly, the invention is to provide a stent used for photodynamic therapy. The said stent can provide a photosensitizer to the portion comprising the tumor and protect the lumen from blocking. The present invention integrates the stent and the photodynamic therapy and provides a stent used for photodynamic therapy. The said stent can provide the photosensitizer to the portion of the tumor and protect the lumen from blocking. Furthermore, the present invention can greatly reduce the needed dosage of the photosensitizer while maintaining the effective concentration of the photosensitizer around tumor cells by the film covering the stent and the photosensitizing layer of the film.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. A stent, for supporting a sidewall of a lumen containing a first fluid, comprising:
a stent body disposed inside the lumen;
a film adapted to be penetrated by a light and covering the stent body; and
a photosensitizing layer disposed on an outer surface of the film, for receiving the light to generate a first object, wherein the film separates the photosensitizing layer and the first fluid.

2. The stent of claim 1, wherein the stent body is a plastic pipe or a metal net pipe.

3. The stent of claim 1, further comprising:
a lubricating layer disposed on the outer surface of the photosensitizing layer, wherein the lubricating layer is hydrophilic and biodegradable.

4. The stent of claim 1, wherein the wavelength of the light received by the photosensitizing layer ranges from 500 nm to 800 nm.

5. The stent of claim 1, wherein the film comprises a porous structure.

6. The stent of claim 1, wherein the photosensitizing layer comprises a photosensitizer used for photodynamic therapies, the first object generated by the photosensitizing layer is singlet oxygen.

7. The stent of claim 1, wherein the film further comprises a light shielding pattern, the light shielding pattern separates the film into a light transmissive region and a light tight region.

8. The stent of claim 1, further comprising a light source fixed with the stent body, for providing the light.

9. The stent of claim 8, further comprising an electrical energy receiver, wherein the electrical energy receiver is coupled to the light source, for transmitting the electrical energy provided from the outside of the lumen by a wireless power supply technology.
